(19) United States
(12) Patent Application Publication
(10) Pub. No.: US 2024/0415769 A1
(43) Pub. Date: Dec. 19, 2024

(54) FORMULATIONS

(71) Applicant: UCB Biopharma SRL, Brussels (BE)

(72) Inventors: Claude PEERBOOM, Brussels (BE); Michaël Joseph Edouard BOONEN, Brussels (BE)

(21) Appl. No.: 18/703,661

(22) PCT Filed: Oct. 20, 2022
(86) PCT No.: PCT/EP2022/079182

§ 371 Date: Apr. 22, 2024

(30) Foreign Application Priority Data
Oct. 21, 2021 (GB) 2115121.2

(51) Int. Cl. A61K 9/08; A61K 47/02; A61K 47/12; A61K 47/18; C07K 16/40

(52) U.S. Cl. CPC A61K 9/08; A61K 47/02; A61K 47/12; A61K 47/183; C07K 16/40

(57) ABSTRACT

The invention relates to the field of pharmaceutical formulations. More particularly it is directed to liquid formulations comprising anti-TG2 antibodies and to methods of producing such formulations. The liquid formulations according to the invention are stable upon storage at a temperature from about 2 to 25° C. for an appropriate period of time.

### FIELD OF INVENTION

The invention relates to the field of pharmaceutical formulations. More particularly it is directed to liquid formulations comprising anti-TG2 antibodies and to methods of producing such formulations. The liquid formulations according to the invention are stable upon storage at a temperature from about 2 to 25° C. for an appropriate period of time.

### BACKGROUND OF THE INVENTION

Tissue transglutaminase (TG2) is an enzyme which forms crosslinks between proteins via epsilon(gamma-glutamyl) lysine bridges. Elevated expression of TG2 leads to aberrant protein cross-linking which has been associated with several pathologies including various types of tissue scarring, the formation of neurofibrillary tangles in several brain disorders and resistance to chemotherapy in some cancers. Various TG2 inhibitors, such as small molecules, silencing RNA or antibodies (e.g. WO2006100679, WO2012146901 or WO2013175229), have been disclosed for the possible treatment of TG2-mediated disorders.

Although antibodies directed to TG2 have been described in the literature, no stable formulations have been proposed so far.

When preparing a pharmaceutical composition comprising a bioactive protein, such as an antibody, said composition must be formulated in such a way that the protein is stable for an appropriate period of time. A loss in activity/stability of the protein may result from chemical or physical instabilities of the protein notably due to denaturation, aggregation or oxidation. The resulting products may thus be pharmaceutically unacceptable. Although the use of excipient(s) is known to increase the stability of a given protein, the stabilizing effects of these excipients is highly dependent of the nature of the excipients and of the bioactive protein itself.

There remains a need for liquid formulations containing an anti-TG2 antibody as an active ingredient, wherein said formulations are stable for an appropriate period of time and suitable for use in injection, such as for intravenous or subcutaneous injection. Said formulations could be useful for administration in the treatment of TG2-mediated disorders or diseases.

### SUMMARY OF THE INVENTION

It is an object of the present invention to provide novel formulations containing an anti-TG2 antibody. More particularly, said formulations are stable liquid formulations containing an anti-TG2 antibody. The invention also provides methods for preparing the liquid formulations according to the present invention. The liquid formulations herein described may be useful for administration in the treatment of TG2-mediated disorders or diseases.

In a first aspect, the invention provides stable liquid formulations comprising or consisting of an anti-TG2 antibody, a buffer which keeps the pH at or about 5.0 to 7.0, a stabilizer (such as an amino acid or a salt) and optionally a polysorbate surfactant. In a preferred embodiment, the buffer is a histidine or a citrate buffer, and the stabilizer is either an amino acid, preferably glycine, or a salt, preferably NaCl. In a further preferred embodiment, the buffer keeps the pH at or about 5.5 to 6.5. In a further preferred embodiment, the anti-TG2 antibody is in an amount of or of about 10 mg/mL to or to about 200 mg/mL. Preferably, the anti-TG2 antibody comprises a light chain variable region as defined in SEQ ID NO: 1 and a heavy chain variable region as defined in SEQ ID NO: 2.

In a second aspect, the invention provides a method for manufacturing a stable liquid formulation of an anti-TG2 antibody, comprising the steps of: forming a mixture of anti-TG2 antibody, together with a buffer, a stabilizer (such as an amino acid or a salt) and optionally a polysorbate surfactant.

In a preferred embodiment, the buffer is a histidine or a citrate buffer, and the stabilizer is either an amino acid, preferably glycine, or a salt, preferably NaCl. In a preferred embodiment the buffer keeps the pH at or about 5.0 to 7.0, and more particularly at or about 5.5 to or to about 6.5.

Preferably, the anti-TG2 antibody comprises a light chain variable region as defined in SEQ ID NO: 1 and a heavy chain variable region as defined in SEQ ID NO: 2.

In a third aspect, here is provided an article of manufacture for pharmaceutical or veterinary use, comprising a container comprising the stable liquid formulation according to the invention.

In a fourth aspect, the invention provides the stable liquid formulation according to the invention for use in therapy

In a fifth aspect, the invention provides a method for treating a disease or disorder by administering the stable liquid formulation according to the invention.

### Definitions

The term “about” means approximately or nearly, and in the context of a numerical value set forth herein preferably designates+/−10% around the numerical value recited or claimed.
When a range of values is recited or claimed, the range is intended to be inclusive of the recited values.
The term “anti-TG2 antibody”, as used herein, is intended to be an antibody molecule which binds the Tissue transglutaminase (TG2) protein, an enzyme which forms crosslinks between proteins via epsilon(gamma-glutamyl) lysine bridges. Examples of such antibodies are described in WO2013175229. Without any limitation, an anti-TG2 antibody that can be used according to the present invention comprises for instance a light chain variable region as defined in SEQ ID NO: 1 and a heavy chain variable region as defined in SEQ ID NO: 2.
The term “antibody” as used herein includes, but is not limited to, monoclonal antibodies, polyclonal antibodies and recombinant antibodies that are generated by recombinant technologies as known in the art. “Antibody” include antibodies of any species, in particular of mammalian species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old-World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” refers to both glycosylated and aglycosylated antibodies. Furthermore, the term “antibody” as used herein not only refers to full-length antibodies, but also refers to antibody fragments, more particularly to antigen-binding fragments thereof. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe®) or Fab-(dsscFv)2 (also referred to as TrYbe®, see WO2015/197772 for instance). Antibody molecules as defined above, including antigen-binding fragments thereof, are known in the art.
The term “stability”, as used herein, refers to the physical, chemical, and conformational stability of the anti-TG2 antibody formulations according to the present invention (and including maintenance of biological potency). Instability of said antibody formulation may be caused by chemical degradation or aggregation of the antibody to form higher order polymers, deglycosylation, modification of glycosylation, oxidation or any other structural modification that reduces at least one biological activity of the antibody.
The term “stable formulation” refers to a formulation in which the protein of interest (herein an anti-TG2 antibody) essentially retains its physical, chemical and biological properties upon storage. In order to measure the stability of an antibody in a formulation, various analytical methods are well within the knowledge of the skilled person (see some examples in the example section). Stability is typically assessed at a selected temperature (for instance −70° C., 2-8° C., 25° C., 35° C. or more) for a selected time period (e.g. 3 months, 6 months, 12 months or more). As an antibody, once formulated, is typically stored in the fridge (typically 2-8° C.) or at room temperature (typically 15-25° C.) before being administered to a patient, it is important that said formulated antibody is stable over time at least at a temperature range of 2 to 25° C., as herein shown for example at 2-8° C. and 25° C. Various values can be used to conclude about stability over a given time period, such as (and not limited to): 1) not less than 90% of monomeric form of the antibody, 2) no more than 10% of alteration of the monomeric form of the antibody (in comparison of the initial data), 3) no more than 5% of High Molecular Weight Species (HMW or HMWS; also herein referred to as aggregates), or 4) no more than +/−0.2 unit variation of the pH (in comparison of the initial data).
The term “stabilizing agent”, “stabilizer” or “isotonicity agent”, as used herein, is a compound that is physiologically tolerated and imparts a suitable stability/tonicity to a formulation. It prevents notably the net flow of water across cell membranes that are in contact with the formulation. Compounds such as glycerin, are commonly used for such purposes. Other suitable stability agents include, but are not limited to, amino acids or proteins (e.g. glycine or albumin), salts (e.g. sodium chloride), and sugars (e.g. dextrose, mannitol, sucrose and lactose).
The term “buffer”, as used herein, refers to solutions of compounds that are known to be safe in formulations for pharmaceutical or veterinary use and that have the effect of maintaining or controlling the pH of the formulation in the pH range desired for the formulation. Acceptable buffers for controlling pH at a moderately acidic pH to a moderately basic pH include, but are not limited to, phosphate, acetate, citrate, arginine, histidine and TRIS (2-amino-2-hydroxymethyl-1,3, -propanediol, the term includes any pharmacologically acceptable salt thereof) buffers.
The term “surfactant”, as used herein, refers to a soluble compound that can be used notably to increase the water solubility of hydrophobic, oily substances or otherwise increase the miscibility of two substances with different hydrophobicities. For this reason, these polymers are commonly used in industrial applications, cosmetics, and pharmaceuticals. They are also used as model systems for drug delivery applications, notably in order to modify the absorption of the drug or its delivery to the target tissues. Well known surfactants include polysorbates (polyoxyethylene derivatives; Tween) as well as poloxamers (i.e. copolymers based on ethylene oxide and propylene oxide, also known as Pluronics®).
The term “vial” or “container”, as used herein, refers broadly to a reservoir suitable for retaining the anti-TG2 antibody formulation in liquid form. Examples of a vial that can be used in the present invention include an ampoule, a tube, a bottle, a syringe (such as a pre-filled syringe), cartridges, or other such reservoir suitable for delivery of the anti-TG2 antibody formulation to the patient via injection, preferably via intravenous or subcutaneous injection.
The term “solvent”, as used herein, refers to a liquid solvent either aqueous or nonaqueous. The selection of the solvent depends notably on the solubility of the drug compound on said solvent and on the mode of administration. Aqueous solvent may consist solely of water, or may consist of water plus one or more miscible solvents, and may contain dissolved solutes such as sugars, buffers, salts or other excipients. The more commonly used non-aqueous solvents are the short-chain organic alcohols, such as, methanol, ethanol, propanol, short-chain ketones, such as acetone, and poly alcohols, such as glycerol. According to the present invention, the preferred solvent is an aqueous solvent such as water or a saline solvent.
The term “treating” or “treatment” of a disease state includes: (i) inhibiting the disease state, i.e. arresting the development of the disease state or its clinical symptoms, or (ii) relieving the disease state, i.e. causing temporary or permanent regression of the disease state or its clinical symptoms.
The term “preventing” or “prevention” of a disease state includes causing the clinical symptoms of the disease state not to develop in a subject that may be exposed to or predisposed to the disease state, but does not yet experience or display symptoms of the disease state.- In all the embodiments of the invention, “pharmaceutical composition” can also be referred to as “stable pharmaceutical composition” without any differentiation.

### DETAILED DESCRIPTION OF THE INVENTION

The invention is based on the combination of a stabilizer selected from the group consisting of glycine or NaCl, and a buffer solution, such as histidine or citrate buffer keeping the pH between 5.0 to 7.0 for preparing a suitable pharmaceutical composition for human use of an anti-TG2 antibody without affecting the processability of the pharmaceutical composition and the long-term stability of the antibody. It is a finding from the inventors that the pharmaceutical compositions according to the invention are stable overtime, in particular when stored at about 2-25° C., as shown in the examples section at 2-8° C. and 25° C.

The main object of the present invention is a stable liquid formulation comprising or consisting of an anti-TG2 antibody, a buffer keeping the pH between about 5.0 and about 7.0, and a stabilizer. In a preferred embodiment, the buffer is a histidine buffer or a citrate buffer, and stabilizer selected from the group consisting of glycine or NaCl. Optionally, the formulation may further comprise a surfactant, such as a polysorbate surfactant.

The invention further provides a method for manufacturing any of the herein described stable liquid formulations of an anti-TG2 antibody, wherein the method comprises the steps of combining the anti-TG2 antibody, together with a buffer, a stabilizer and optionally a surfactant, such as a polysorbate surfactant. Said step is typically performed by buffer exchange according to conventional procedures. As an example, in order to prepare a suitable stable formulation, a given amount of an anti-TG2 antibody is buffer exchanged with 1) a citrate or a histidine buffer which keeps the pH at or about 5.0 to 7.0, 2) a stabilizer (preferably selected from the group consisting of glycine or NaCl). Should the formulation comprise a surfactant, it is preferably added after the buffer exchange step. After buffer exchange, the formulation is filtered (final filtration). Depending on the target concentration for the antibody, the formulation can be concentrated between the step of buffer exchange and the final filtration. Each of these compounds (i.e. the anti-TG2 antibody, the buffer, the stabilizer and optionally the surfactant) can be used according to the concentrations, pH, and/or ratios herein described. The resulting mixture is then dispensed into vials. Variations of this process will be recognized by one of ordinary skill in the art.

The invention also provides an article of manufacture, for pharmaceutical or veterinary use, comprising a container comprising any of the herein described stable liquid formulation, said formulation comprising or consisting of anti-TG2 antibody, a buffer, a stabilizer, and optionally a surfactant. Each of these compounds (i.e. the anti-TG2 antibody, the buffer, the stabilizer and optionally the surfactant) can be used according to the concentrations, pH, and/or ratios herein described.

Also described, a packaging material providing instructions for use.

Preferably, the anti-TG2 antibody to be used according to the invention as a whole comprises (see also Table A):
1) a light chain variable domain having the sequence as defined in SEQ ID NO: 1 and a heavy chain variable domain having the sequence as defined in SEQ ID NO: 2
2) a light chain variable domain having at least 80% identity or similarity, preferably 90% identity or similarity to the sequence as defined in SEQ ID NO: 1 and a heavy chain variable domain having at least 80% identity or similarity, preferably 90% identity or similarity to the sequence as defined in SEQ ID NO: 2.
3) a light chain having the sequence as defined in SEQ ID NO: 3 and a heavy chain having the sequence as defined in SEQ ID NO: 4; or
4) a light chain having at least 80% identity or similarity, preferably 90% identity or similarity to the sequence as defined in SEQ ID NO: 3 and a heavy chain having at least 80% identity or similarity, preferably 90% identity or similarity to the sequence as defined in SEQ ID NO: 4.

TABLE A
Anti-TG2 amino acid sequences

| SEQ ID | Amino acid sequence |
|---|---|
| 1 | DITMTQSPSSLSASVGDRVTITCKASQDINSYLTWFQQKPGKA |
| | PKILIYLVNRLVDGVPSRFSGSGSGQDYALTISSLQPEDFATY |
| | YCLQYDDFPYTFGQGTKVEIK |
| 2 | EVQLLESGGGLVQPGGSLRLSCAASGFTLSTHAMSWVRQAPGK |
| | GLEWVATISSGGRSTYYPDSVKGRFTISRDNSKNTLYLQMNSL |
| | RAEDTAVYFCARLISTYWGQGTLVTVSS |
| 3 | DITMTQSPSSLSASVGDRVTITCKASQDINSYLTWFQQKPGKA |
| | PKILIYLVNRLVDGVPSRFSGSGSGQDYALTISSLQPEDFATY |
| | YCLQYDDFPYTFGQGTKVEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| 4 | EVQLLESGGGLVQPGGSLRLSCAASGFTLSTHAMSWVRQAPGK |
| | GLEWVATISSGGRSTYYPDSVKGRFTISRDNSKNTLYLQMNSL |
| | RAEDTAVYFCARLISTYWGQGTLVTVSSASTKGPSVFPLAPCS |
| | RSTSESTAALGCLVKDYFPEPVTVSWNSGALTSGVHTFPAVLQ |
| | SSGLYSLSSVVTVPSSSLGTKTYTCNVDHKPSNTKVDKRVESK |
| | YGPPCPPCPAPEFLGGPSVFLFPPKPKDTLMISRTPEVTCVVV |
| | DVSQEDPEVQFNWYVDGVEVHNAKTKPREEQFNSTYRVVSVLT |
| | VLHQDWLNGKEYKCKVSNKGLPSSIEKTISKAKGQPREPQVYT |
| | LPPSQEEMTKNQVSLTCLVKGFYPSDIAVEWESNGQPENNYKT |
| | TPPVLDSDGSFFLYSRLTVDKSRWQEGNVFSCSVMHEALHNHY |
| | TQKSLSLSLGK |

In the context of the invention as a whole, the amount of anti-TG2 antibody in the formulations is preferably from or from about 10 mg/mL to or to about 200 mg/mL, preferably from or from about 30 mg/mL to or to about 180 mg/mL, or preferably from or from about 50 mg/mL to or to about 150 mg/mL, such as 50, 55, 60, 65, 70, 75, 80, 85, 90, 95, 100, 105, 110, 115, 120, 125, 130, 135, 140, 145 or 150 mg/mL. Alternatively, the anti-TG2 antibody is present in the protein formulations in an amount expressed in terms of weight per 100 mL (% w/v). In such a case, the anti-TG2 antibody comprised in the formulations according to the present invention as a whole can be present in an amount of about 1 to or to about 20% w/v, preferably in an amount of about 3 to or to about 18% w/v, or preferably in an amount of about 5 to or to about 15% w/v such as 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5 or 15.0% w/v. The anti-TG2 antibody may for instance (but not limited to) comprise a light chain variable region as defined in SEQ ID NO: 1 and a heavy chain variable region as defined in SEQ ID NO: 2.

Preferable buffers according to the present invention as a whole are histidine (preferably L-Histidine) or citrate buffers and keep the pH comprised between about 5.0 and about 7.0, preferably comprised between about 5.2 and about 6.0, such as 5.2, 5.3, 5.4, 5.5, 5.6, 5.7, 5.8, 5.9 and 6.0 for a histidine buffer and preferably comprised between about 6.2 and about 7.0, such as 6.2, 6.3, 6.4, 6.5, 6.6, 6.7, 6.8, 6.9 and 7.0 for a citrate buffer. Even more preferably the pH is at or about 5.5 for a histidine buffer and at or about 6.5 for a citrate buffer. In all the embodiments of the present invention, unless otherwise indicated, the pH value was measured at room temperature and it is preferably within ±0.1 or ±0.2 of the targeted pH unit (e.g. 5.5±0.1 or 5.5±0.2 for a histidine buffer and at or about 6.5±0.1 or 6.5±0.2 for a citrate buffer).

In the context of the invention as a whole, the buffer concentration is preferably at or about 10 to 100 mM. In a preferred embodiment, the concentration of the buffer is at or about 20 to or to about 80 or even preferably about 40 to about 60 mM, such as 40, 45, 50, 55 or 60 mM. Preferably, the concentration of the buffer is at or about 50 mM.

In the context of the invention as a whole, the stabilizer is selected from the group consisting of glycine (preferably L-glycine) or NaCl. Should the stabilizer be glycine, its concentration is preferably at or at about 150 mM to or to about 350 mM, preferably at or at about 200 to or to about 300 mM or even preferably at or at about 220 to or to about 280 mM, such as 220, 230, 240, 250, 260, 270 or 280 mM. Should the stabilizer be NaCl, its concentration is preferably at or at about 100 mM to or to about 200 mM, preferably at or at about 125 to or to about 175 mM, such as 125, 130, 135, 140, 145, 150, 155, 160, 165, 170 and 175 mM.

In the context of the invention as a whole, a surfactant can be optionally present. When present, the surfactant is preferably a polysorbate surfactant such as polysorbate 20 (PS20 also known as Tween® 20) or polysorbate 80 (PS80 also known as Tween® 80). Preferably the surfactant is present in the formulation in an amount of or of about 0.01 to or to about 5 mg/mL, more preferably of or of about 0.01 to or to about 1 mg/mL, more particularly of or of about 0.1 to or to about 0.6 mg/mL, such as 0.1, 0.15, 0.2, 0.25, 0.3, 0.35, 0.4, 0.45, 0.5, 0.55 or 0.6 mg/mL. Alternatively, the polysorbate surfactant is preferably present in the protein formulation in an amount expressed in terms of % weight per 100 mL (% w/v). In such a case, the polysorbate surfactant comprised in the formulation according to the present invention as a whole can be present in an amount of 0.001 to 0.5% w/v, preferably from 0.01 to 0.1% w/v, or even preferably from 0.01 to 0.06% w/v such as 0.01, 0.015, 0.02, 0.025, 0.03, 0.035, 0.04, 0.045, 0.05, 0.055 or 0.06% w/v.

In a preferred embodiment, the stable liquid formulations according to the present invention as a whole comprise or consist of an anti-TG2 antibody at or at about 50 to 200 mg/mL, about 10 to about 100 mM of histidine at pH about 5.5 (or about 10 to about 100 mM citrate buffer at pH about 6.5), about 150 to about 350 mM of glycine or about 100 to about 200 mM of NaCl, and optionally a surfactant (such as a polysorbate surfactant) at about 0.001 mg/mL to about 0.5 mg/mL. Alternatively, the stable liquid formulations comprise or consist of an anti-TG2 antibody at about 5.0 to about 20% w/v, about 10 to about 100 mM of histidine at pH about 5.5 (or about 10 to about 100 mM citrate buffer at pH about 6.5), about 150 to about 350 mM of glycine or about 100 to about 200 mM of NaCl, and optionally 0.001 to 0.5% w/v of surfactant (such as a polysorbate surfactant).

As a specific example (but not limited to), herein is provided a stable liquid formulation comprising or consisting of an anti-TG2 antibody at about 100 mg/mL, about 50 mM of histidine buffer keeping the pH at about 5.5, about 250 mM of glycine and optionally PS80 at about 0.02-0.06% w/v. As a further specific example (but not limited to), herein provided is a stable liquid formulation comprising or consisting of an anti-TG2 antibody at about 100 mg/mL, about 50 mM of histidine buffer keeping the pH at or at about 5.5, about 150 mM of NaCl and optionally PS80 at about 0.02-0.06% w/v. In an even further example (but not limited to), it is provided a stable liquid formulation that comprises or consists of an anti-TG2 antibody at about 100 mg/mL, about 50 mM of citrate buffer keeping the pH at about 5.5, about 250 mM of glycine and optionally PS80 at about 0.02-0.06% w/v. The anti-TG2 antibody may for instance comprise a light chain variable region as defined in SEQ ID NO: 1 and a heavy chain variable region as defined in SEQ ID NO: 2.

Preferably the formulations of the invention retain at least 80% of the biological activity of the anti-TG2 antibody at the time of formulation and/or packaging over a period of at least 12 months, preferably at least 24 months or even preferably at least 36 months or even more preferably at least 48 months (before the first use). The anti-TG2 antibody activity may be measured according to routine methods such as Elisa or cell-based assays.

Additional excipients for use within the pharmaceutical compositions according to the invention include, but are not limited to, viscosity enhancing agents, bulking agents, solubilising agents or combinations thereof.

The present invention also provides for a container comprising the pharmaceutical composition according to the invention. In particular, the container may be, without any limitations, a vial, an ampoule, a tube, a bottle or a syringe (such as a pre-filled syringe) comprising the pharmaceutical composition.

The container may be part of a kit-of-parts comprising one or more containers comprising the pharmaceutical compositions according to the invention and delivery devices such as a syringe, pre-filled syringe, an autoinjector, a needleless device, an implant or a patch, or other devices for parental administration and instructions of use.

The liquid formulations of the invention may be kept for at least about 12 months to about 48 months. Under preferred storage conditions, before the first use, the formulations are kept away from bright light (preferably in the dark), at temperature from about 2 to 25° C., e.g. at room temperature (at or about 25° C.) or at 2-8° C. (see following examples). Said formulations minimize the loss of active principle, i.e. an anti-TG2 antibody. It has also been found that said formulations are less prone to acidification or to degradation such as formation of protein aggregates.

The present invention provides stable liquid formulations of anti-TG2 antibody for use in therapy. For instance, the stable liquid formulations of anti-TG2 antibody herein described, are suitable for pharmaceutical or veterinary use. The present invention also provides a method for treating a disease or disorder by administering stable liquid formulations of anti-TG2 antibody.

The stable liquid formulation comprising anti-TG2 antibody according to the present invention, can be administered for improving or for treating TG2-mediated disorders or diseases. Such TG2-mediated disorders or diseases can for instance be selected from the group consisting of Celiac disease, abnormal wound healing, scarring, keloids and hypertrophic scars, ocular scarring, inflammatory bowel disease, macular degeneration, Grave's ophthalmopathy, drug-induced ergotism, psoriasis, fibrotic diseases or fibrosis-related diseases, atherosclerosis, restenosis, inflammatory diseases, autoimmune diseases, neurodegenerative/neurological diseases (e.g. Huntington's Disease, Alzheimer's disease, Parkinson's disease, polyglutamine disease, spinobulbar muscular atrophy, dentatorubral-pallidoluysian atrophy, spinocerebellar ataxias 1, 2, 3, 6, 7 and 12, rubropallidal atrophy, spinocerebellar palsy), and/or cancer (e.g. glioblastomas such as glioblastoma in Li-Fraumeni syndrome and sporadic glioblastoma, malignant melanomas, pancreatic ductal adenocarcinomas, myeloid leukemia, acute myelogenous leukemia, myelodysplasia syndrome, myeloproliferative syndrome, gynaecological cancer, Kaposi's sarcoma, Hansen's disease, collagenous colitis).

The pharmaceutical composition according to the invention may be administered in a therapeutically effective amount. The term “therapeutically effective amount” as used herein refers to an amount of a therapeutic agent (i.e. an antibody) needed to treat, improve or prevent a TG2-mediated disorder or disease, or to exhibit a detectable therapeutic, pharmacological or preventative effect. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

For the treatment of the above diseases and/or disorders, the appropriate dosage will vary depending upon, for example, the particular antibody to be employed, the subject treated, the mode of administration and the nature and severity of the condition being treated. In a particular embodiment, the pharmaceutical composition according to the invention is administered by intravenous or subcutaneous route. When administered via intravenous injection, it may be administered as a bolus injection or as a continuous infusion. The pharmaceutical composition according to any of the embodiments of the invention may also be administered by intramuscular injection. Depending on the administration mode, the formulations herein described can be diluted in a solvent (such as NaCl) before use. The pharmaceutical composition may be injected using a syringe, an injection device such as an autoinjector, a needleless device, an implant and a patch. The liquid pharmaceutical formulation of the invention is suitably administered to the patient at one time or over a series of treatments and may be administered to the patient at any time from diagnosis onwards; it may be administered as the sole treatment or in conjunction with other drugs or therapies useful in treating the conditions as described herein before.

The anti-TG2 antibody may be the sole active ingredient in the liquid pharmaceutical formulation. Alternatively, the antibody may be administered in combination, e.g. simultaneously, sequentially or separately, with one or more other therapeutically active ingredients. Active ingredient as employed herein refers to an ingredient with a pharmacological effect, such as a therapeutic effect, at a relevant dose. In some embodiments the antibody in the pharmaceutical composition may be accompanied by other active ingredients including other antibodies or non-antibody ingredients, administered by the same or by a different route of administration, to treat other inflammatory or autoimmune diseases. In one embodiment, the subject is administered, simultaneously or in sequence (before and/or after) other antibody ingredients, such as anti-TNF antibodies or non-antibody ingredients such as small molecule drug molecules.

The following examples are provided to further illustrate the preparation of the formulations and compositions of the invention. The scope of the invention shall not be construed as merely consisting of the following examples.

### EXAMPLES

### Material

Anti-TG2 antibody: the anti-TG2 monoclonal antibody (mAb) that was used in the following examples comprised a light chain variable region as defined in SEQ ID NO: 1 and a heavy chain variable region as defined in SEQ ID NO: 2. It is named mAb1 in the following examples.

### Methods

Proteins concentration: Protein concentration was determined using the UV-Visible spectroscopic method, using the following equation: Concentration (mg/mL)=[(A280)/a×b]

Where A280=Absorbance at 280 nm (AU); a=Mass Extinction Coefficient (1.34 mL mg⁻¹ cm⁻¹); b=Path length (1 cm)

Thermal stability: Proteins can be unfolded at specific temperature range. This temperature, also known as melting temperature (Tm), is an intrinsic parameter describing thermal stability of proteins. Tm is the peak temperature during protein unfolding. Capillary Differential Scanning Calorimetry (DSC) was used to determine Tm for mAb1. The autosampler was used to fill the wells with about 300 μL of buffer and mAb1 sample, respectively. The reference wells were filled only with buffer. DSC scans were analysed using MicroCal Origin® software. After subtraction of the respective buffer scan, protein scans were normalized against the protein concentration. The Tm values were determined using the “Pick Peak” tool in MicroCal Origin® software.

Diffusion behaviour: Dynamic light scattering (DLS) was used for that purpose according to standard methods. Among the different parameters that were evaluated, Main Species Peak Diameter and Width: Each peak represents a distinct and resolvable species or population of particles. Peak diameter represents the hydrodynamic diameter (expressed in nm) of the resolved species. The peak width is a measure of the polydispersity of the population particles in a peak. “Peak 1” is regarded as the main species peak and typically corresponds to the non-aggregated forms of a protein.

Aggregations and fragmentations: Size Exclusion chromatography (SEC), such as Size Exclusion High-Performance Liquid Chromatography (SE-HPLC), was used according to standard methods. The percentage peak area values for the main species, as well as the species that elute before and after the main peak, designated high molecular weight (HMW) and low molecular weight (LMW) species, respectively, were evaluated.

Purity in reduced or non-reduced conditions: The purity in reduced or non-reduced conditions (IgG monomer) was evaluated using Capillary Gel Electrophoresis (CGE) or on-chip based Electrophoresis (Bioanalyzer) according to standard methods (separating proteins based on differences in their hydrodynamic size under denaturing conditions). The various mAb1 species were detected by a photo diode array (PDA) detector at 220 nm.

Acid and basic species: The presence of acid and basic species was evaluated using Isoelectric Capillary Electrophoresis (iCE) according to standard iCE methods (separating proteins based on differences in their charges). Typically, peaks eluting before the main peak are labelled as acidic species and those eluting post main peak are labelled as basic species.

Osmolality: Osmolality was assessed according to standard methods, using an Osmette XL 5007 osmometer, calibrated with deionized water (zero mOsm/kg), 100 mOsm/kg, 500 mOsm/kg and 1500 mOsm/kg standard solutions prior to the analysis of the mAb1 samples.

pH: pH was evaluated according to standard methods, using a pH meter equipped with a temperature compensating electrode.

Viscosity: Viscosity measurements were performed according to standard methods, using the Rheosense MicroVisc®. The viscosity measurements were performed in quadruplicate, and the averaged dynamic viscosity result is reported.

### Example 1—Preliminary Screening

### 1.1 Buffers and pH Screening

Five different buffer types, each at two or three different pH were evaluated with regard to their effect on thermal and physical stability of mAb1. The mAb1 samples were buffer exchanged, according to standard methods into the buffers listed in Table 1. The target protein concentration for this preliminary screening was 2 mg/mL. The prepared samples were analysed for pH, protein concentration and thermal and conformational stability.

The pH of each sample was determined following the buffer exchange process. Each sample pH was within 0.2 pH unit from the targeted pH value (data not shown). The mAb1 concentration of each sample was determined using UV spectroscopy (see Table 1). Overall, the recoveries ranged from 52% to 127%. In view of the variability of these results, no clear trend was identified.

TABLE 1
mAb1 concentration and percent recovery
for each of the tested conditions

| | | Average | % |
|---|---|---|---|
| Buffer | pH | concentration | recovery |
| Acetate (20 mM)(Ace) | 4.5 | 2.06 | 73 |
| | 5.5 | 2.01 | 97 |
| Succinate (20 mM)(Suc) | 4.5 | 2.07 | 70 |
| | 5.5 | 1.98 | 82 |
| | 6.5 | 2.05 | 101 |
| Citrate (20 mM)(Cit) | 5.0 | 2.03 | 87 |
| | 6.0 | 2.03 | 77 |
| | 7.0 | 2.07 | 96 |
| Histidine (20 mM)(His) | 6.0 | 2.05 | 80 |
| | 6.5 | 2.02 | 52 |
| | 7.0 | 1.99 | 70 |
| Phosphate (20 mM)(Pho) | 6.0 | 2.02 | 69 |
| | 6.5 | 2.05 | 127 |
| | 7.0 | 2.03 | 64 |

The mAb1 samples were then analysed by DLS for diffusion behaviour and DSC for aggregate species (data not shown). While the Tm of the formulated samples was relatively similar, ranging from 75.3° C. to 77.6° C., the onset temperatures were more informative. Formulations with pH<5.5 displayed onset temperatures ≤53° C. In addition, it was observed that all formulations with pH<5.5 displayed three thermal transitions, but only two transitions for the other formulations. In view of the data, buffer systems with pH ≥5.5 seem more optimal.

Based on these results, phosphate buffer was removed from further evaluation due to lack of advantage over other buffering systems and the potential issues with freeze/thaw. Further, based on the DSC data, pH 5.5 was selected for excipient screening.

### 1.2. Excipient Screening

Based on the buffers and pHs screen results, four buffers at pH 5.5 were evaluated in combination with various excipients (See Table 2) to determine their ability to confer stability to mAb1. mAb1 samples were buffer exchanged into the buffers listed in the Table 6, according to standard methods. The targeted mAb1 concentration was 2 mg/mL. The pH and percent recovery of each mAb1 sample were determined, as well as the thermal and conformational stability of mAb1 in the various buffers (using DSC and DLS; data not shown).

TABLE 2
Excipient Screening

| | | | Average | |
|---|---|---|---|---|
| | | | Concentration | % |
| Buffer | pH | Excipient | (mg/ml) | Recovery |
| Acetate | 5.5 | 250 mM Glycine (Gly) | 2.0 | 86 |
| (20 mM) | | 250 mM Arginine (Arg) | 1.9 | 57 |
| | | 150 mM NaCl | 1.9 | 84 |
| | | 250 mM Sorbitol (Sor) | 2.0 | 93 |
| | | 250 mM Sucrose (Suc) | 1.9 | 92 |
| Succinate | 5.5 | 250 mM Glycine | 1.9 | 91 |
| (20 mM) | | 250 mM Arginine | 2.0 | 89 |
| | | 150 mM NaCl | 1.9 | 90 |
| | | 250 mM Sorbitol | 1.9 | 90 |
| | | 250 mM Sucrose | 1.9 | 85 |
| Citrate | 5.5 | 250 mM Glycine | 1.9 | 90 |
| (20 mM) | | 250 mM Arginine | 1.9 | 90 |
| | | 150 mM NaCl | 1.9 | 91 |
| | | 250 mM Sorbitol | 1.9 | 91 |
| | | 250 mM Sucrose | 2.0 | 93 |
| Histidine | 5.5 | 250 mM Glycine | 1.9 | 91 |
| (20 mM) | | 250 mM Arginine | 1.9 | 60 |
| | | 150 mM NaCl | 1.9 | 89 |
| | | 250 mM Sorbitol | 1.9 | 57 |
| | | 250 mM Sucrose | 1.9 | 91 |

The pH of each sample was determined following the buffer exchange process. With the exception of three formulations (acetate buffer with either glycine, sodium chloride (NaCl) or sorbitol), each pH was within 0.2 unit of the target pH, 5.5 (data not shown). The pH for acetate buffer with glycine, NaCl and sorbitol were 5.8, 5.8 and 5.9, respectively.

The mAb1 concentration of each sample was determined using UV spectroscopy (see Table 2). Three samples (acetate/arginine, histidine/arginine, and histidine/sorbitol) showed poor recoveries, ≤60%. The remaining formulations displayed reasonable recoveries of ≥84%. The variations amongst these formulations did not suggest a clear trend.

mAb1 samples were then analysed by DLS (data not shown). All samples containing sucrose exhibited a characteristic sucrose impurity peak, preventing any observations. Overall, the results did not indicate a clear trend. It is noted that the succinate/sorbitol sample displayed an aggregate species that was not observed in other formulations with sorbitol. In addition, the succinate/NaCl sample also presented the highest monomer width in comparison to all other samples.

DSC results are presented in Table 3. Whatever the buffer system, formulations with glycine displayed higher onset and TM_{Final} temperature than those with arginine. NaCl containing formulations displayed higher onset temperature than arginine in all but the histidine formulations. Sorbitol and sucrose exhibited comparable onset temperatures, within 1° C. of each other.

TABLE 3
DSC Results

| Buffer | Excipient | Onset (° C.) | Tm_{final} (° C.) | Tm₁ (° C.) |
|---|---|---|---|---|
| Acetate | Glycine | 58 | 76.2 | 69 |
| | Arginine | 56 | 76.2 | 63 |
| | NaCl | 60 | 76.8 | 67 |
| | Sorbitol | 62 | 76.7 | 70 |
| | Sucrose | 62 | 77.9 | 70 |
| Succinate | Glycine | 59 | 77.9 | 67 |
| | Arginine | 56 | 76.3 | 63 |
| | NaCl | 58 | 76.8 | 65 |
| | Sorbitol | 60 | 77.5 | 67 |
| | Sucrose | 61 | 77.8 | 68 |
| Citrate | Glycine | 59 | 77.7 | 67 |
| | Arginine | 57 | 76.6 | 64 |
| | NaCl | 58 | 76.9 | 66 |
| | Sorbitol | 59 | 77.4 | 67 |
| | Sucrose | 60 | 77.8 | 67 |
| Histidine | Glycine | 57 | 77.5 | 65 |
| | Arginine | 56 | 75.2 | 63 |
| | NaCl | 53 | 75.6 | 62 |
| | Sorbitol | 56 | 77.5 | 65 |
| | Sucrose | 55 | 77.7 | 65 |

In summary, DSC data showed that glycine and NaCl were preferred over arginine. Therefore, arginine was removed from further evaluation. Both the DLS and DSC data indicated that sucrose and sorbitol were comparable. However, the characteristic sucrose impurity peak made detection of protein aggregation problematic. As such, sorbitol was preferred, and sucrose was not selected for further evaluation. Lastly, since succinate/sorbitol sample contained aggregation species not observed in any other sorbitol samples, succinate was not chosen for further evaluation.

### 1.3. Solubility Study

The solubility of mAb1 was evaluated at different concentrations in the buffers listed in Table 4.

TABLE 4
Solubility Screening

| Buffer | pH | Excipient |
|---|---|---|
| 20 mM Acetate | 5.5 | 250 mM Glycine |
| | | 250 mM Sorbitol |
| | | 150 mM NaCl |
| 20 mM Citrate | 5.5 | 250 mM Glycine |
| | | 250 mM Sorbitol |
| | | 150 mM NaCl |
| 20 mM Histidine | 5.5 | 250 mM Glycine |
| | | 250 mM Sorbitol |
| | | 150 mM NaCl |

mAb1 samples were buffer exchanged into the appropriate buffers according to standard methods. In this study, three mAb1 concentrations were targeted based on volume reduction: 100 mg/mL, 150 mg/mL and 200 mg/mL. The concentration, percent recovery and visual observation results of each sample are presented in Table 5.

The percent recoveries were calculated based on the determined concentration of the preceding evaluation. Within the scope of this solubility study, the visual observation designation “clear” is considered to be equivalent to “clear, colourless”. All formulations achieved the targeted concentrations of 100 mg/mL and 150 mg/ml, with percent recoveries ranging respectively from 54% to 79% and 59% to 98%. All samples were observed to be clear and colourless (CC). All samples were further concentrated to the 200 mg/mL target. The acetate/glycine and acetate/sorbitol samples reached 178 mg/mL and 162 mg/mL, respectively. All other samples achieved concentrations >180 mg/mL with percent recoveries >80%. At these high concentrations, all samples were observed to be clear and gelatinous (CG), except the histidine/NaCl sample which appeared as clear and viscous (CV). Overall, the recoveries did not indicate a clear trend across all the concentrations.

TABLE 5
Protein Concentration and Visual Observation

| | | Target: 100 mg/mL | | | Target: 150 mg/mL | | | Target: 200 mg/mL | | |
|---|---|---|---|---|---|---|---|---|---|---|
| | | Avg | | | Avg | | | Avg | | |
| | | Conc | % | | Conc | % | | Conc | % | |
| Buffer | Excipient | mg/mL | Rec | Obs. | mg/mL | Rec | Obs. | mg/mL | Rec | Obs. |
| Acetate | Glycine | 104 | 72 | CC | 161 | 62 | CC | 178 | 59 | CG |
| Acetate | Sorbitol | 99 | 79 | CC | 140 | 70 | CV | 162 | 72 | CG |
| Acetate | NaCl | 110 | 76 | CC | 170 | 66 | CC | 215 | 84 | CG |
| Citrate | Glycine | 109 | 54 | CC | 159 | 59 | CV | 199 | 94 | CG |
| Citrate | Sorbitol | 101 | 60 | CC | 159 | 78 | CV | 190 | 99 | CG |
| Citrate | NaCl | 106 | 63 | CC | 176 | 69 | CV | 211 | 84 | CG |
| Histidine | Glycine | 105 | 72 | CC | 162 | 67 | CV | 188 | 96 | CG |
| Histidine | Sorbitol | 105 | 62 | CC | 162 | 98 | CV | 207 | 81 | CG |
| Histidine | NaCl | 100 | 79 | CC | 159 | 60 | CV | 203 | 106 | CV |

CC = Clear, colourless;
CV = Clear, viscous;
CG = Clear, Gelatinous;
Avg Conc = average concentration;
% rec = % recovery;
Obs = visual observation

### Example 2—DOE (Design of Experiments) Study

Based on the preliminary study performed according to example 1, a DOE was prepared to evaluate buffer type (focusing on histidine and citrate buffers which were more promising), buffer strength, pH and excipient (see Table 6).

TABLE 6
DOE Design Plan

| Sample | | PS80 | | Buffer Strength | |
|---|---|---|---|---|---|
| # | pH | (% w/v) | Buffer Type | (mM) | Excipient |
| 1 | 6.5 | 0 | Citrate | 35 | 150 mM NaCl |
| 2 | 5.75 | 0.05 | Citrate | 35 | 75 mM NaCl / 125 mM Glycine |
| 3 | 6.5 | 0.05 | Histidine | 35 | 75 mM NaCl / 125 mM Glycine |
| 4 | 5.0 | 0.1 | Histidine | 35 | 150 mM NaCl |
| 5 | 5.75 | 0 | Histidine | 35 | 250 mM Glycine |
| 6 | 6.5 | 0 | Citrate | 50 | 75 mM NaCl / 125 mM Glycine |
| 7 | 5.0 | 0 | Histidine | 20 | 250 mM Glycine |
| 8 | 5.75 | 0.1 | Citrate | 35 | 250 mM Glycine |
| 9 | 5.75 | 0.05 | Histidine | 35 | 250 mM Glycine |
| 10 | 5.75 | 0.1 | Histidine | 50 | 75 mM NaCl / 125 mM Glycine |
| 11 | 6.5 | 0.1 | Histidine | 50 | 150 mM NaCl |
| 12 | 5.75 | 0.1 | Citrate | 20 | 150 mM NaCl |
| 13 | 5.75 | 0 | Citrate | 35 | 75 mM NaCl / 125 mM Glycine |
| 14 | 6.5 | 0 | Histidine | 50 | 250 mM Glycine |
| 15 | 5.75 | 0.05 | Histidine | 20 | 150 mM NaCl |
| 16 | 5.0 | 0.1 | Histidine | 50 | 250 mM Glycine |
| 17 | 5.0 | 0.1 | Histidine | 20 | 75 mM NaCl/ 125 mM Glycine |
| 18 | 6.5 | 0.1 | Histidine | 20 | 250 mM Glycine |
| 19 | 5.75 | 0 | Histidine | 50 | 150 mM NaCl |
| 20 | 6.5 | 0.05 | Citrate | 35 | 150 mM NaCl |
| 21 | 6.5 | 0.1 | Citrate | 20 | 75 mM NaCl / 125 mM Glycine |
| 22 | 5.0 | 0.05 | Histidine | 35 | 150 mM NaCl |
| 23 | 5.0 | 0.05 | Citrate | 20 | 75 mM NaCl / 125 mM Glycine |
| 24 | 5.0 | 0 | Citrate | 50 | 250 mM Glycine |
| 25 | 5.75 | 0.05 | Citrate | 20 | 250 mM Glycine |
| 26 | 5.0 | 0 | Histidine | 50 | 75 mM NaCl / 125 mM Glycine |
| 27 | 5.0 | 0 | Citrate | 20 | 150 mM NaCl |
| 28 | 6.5 | 0 | Histidine | 20 | 150 mM NaCl |
| 29 | 6.5 | 0.1 | Citrate | 50 | 250 mM Glycine |
| 30 | 5.0 | 0.1 | Citrate | 50 | 75 mM NaCl / 125 mM Glycine |
| 31 | 6.5 | 0 | Histidine | 20 | 75 mM NaCl / 125 mM Glycine |
| 32 | 5.75 | 0.05 | Citrate | 50 | 150 mM NaCl |
| 33 | 5.0 | 0.1 | Citrate | 20 | 250 mM Glycine |
| 34 | 5.75 | 0.05 | Histidine | 35 | 75 mM NaCl / 125 mM Glycine |
| 35 | 6.5 | 0 | Citrate | 20 | 250 mM Glycine |
| 36 | 5.0 | 0.1 | Citrate | 50 | 150 mM NaCl |
| 37 | 5.5 | 0 | Histidine | 50 | 250 mM Glycine |

The protein samples were buffer exchanged into the buffer/excipient combinations (see Table 6) according to standard methods. Where appropriate, PS80 surfactant was spiked into the appropriate samples at the specified concentration, following buffer-exchange. Vials of each formulation were placed either at 5° C. or 37° C., for the 4-week- and 7-week-incubation.

Osmolality: The osmolality at TO was within the expected range for all the samples (between 340 and 380 mOsm/kg) (data not shown).

Viscosity: The viscosity was within an acceptable range for all the samples (between 2.5 and 4.0 at TO) (data not shown).

pH: All sample pH values were within 0.2 pH unit of the buffer pH, with the exception of sample #21, which had a pH of 6.2 for the TO, 5° C. 4 Week, 37° C. 4 Week and 5° C. 7 Week time point (data not shown).

Visual observation: The appearance of each of the samples at TO, 4 Week (5° C., 37° C.), and 7 Week (5° C., 37° C.) were evaluated (data not shown). All samples appeared as colourless, clear liquid, free of visible particulates at TO. Samples stored at 5° C. for 4 weeks were all observed to be colourless, clear liquids, free of visible particulates with the exception of samples 1, 2 and 4 which were slightly yellow, clear liquids, free of visible particulates. Samples stored at 37° C. for 4 weeks were all observed to be slightly yellow, clear liquid free of visible particulates with the exception of samples 12, 22, 28, 30, 32, 35 and 36 which were colourless, clear liquid (free of visible particulates). Samples stored at 5° C. for 7 weeks were all observed to be colourless, clear liquid free of visible particulates. Samples stored at 37° C. for 7 weeks were all observed to be slightly yellow, clear liquid (free of visible particulates)

DSC: The DSC results of TO time point are shown in Table 7. Thermograms of samples 8-23 exhibited broader peaks and lower than expected signal.

TABLE 7
DSC results

| Sam- | Onset | Tm1 | Tmfinal |
|---|---|---|---|
| ple # | (° C.) | (° C.) | (° C.) |
| 1 | 60 | 68 | 77.1 |
| 2 | 60 | 67 | 77.9 |
| 3 | 59 | 67 | 77.4 |
| 4 | 48 | 55 | 70.2 |
| 5 | 55 | 64 | 77.5 |
| 6 | 61 | 68 | 77.7 |
| 7 | 53 | 60 | 75.7 |
| 8 | 55 | 66 | 75.0 |
| 9 | 40 | 57 | 68.3 |
| 10 | 51 | 59 | 68.1 |
| 11 | 52 | 62 | 70.3 |
| 12 | 52 | 63 | 71.0 |
| 13 | 51 | 63 | 71.7 |
| 14 | 44 | NA | 59.2 |
| 15 | 50 | NA | 69.3 |
| 16 | 39 | 51 | 64.7 |
| 17 | 43 | NA | 63.8 |
| 18 | 34 | NA | 56.8 |
| 19 | 47 | NA | 66.8 |
| 20 | 49 | 59 | 68.4 |
| 21 | 50 | 62 | 69.5 |
| 22 | 48 | NA | 66.6 |
| 23 | 50 | NA | 66.1 |
| 24 | 53 | 62 | 76.7 |
| 25 | 59 | 68 | 77.7 |
| 26 | 51 | 59 | 73.5 |
| 27 | 55 | 63 | 76.6 |
| 28 | 60 | 67 | 76.8 |
| 29 | 60 | 69 | 77.6 |
| 30 | 55 | 62 | 76.8 |
| 31 | 60 | 68 | 77.0 |
| 32 | 59 | 67 | 77.2 |
| 33 | 58 | 65 | 77.5 |
| 34 | 57 | 64 | 76.7 |
| 35 | 61 | 70 | 77.1 |
| 36 | 61 | 69 | 77.4 |
| 37 | 56 | 63 | 77.0 |

SEC: The SEC results of 4 Week (5° C., 37° C.), and 7 Week (5° C., 37° C.) time points are shown in Table 8.

TABLE 8
SEC Results

| | 4 weeks | | | | | | 8 weeks | | | | | |
|---|---|---|---|---|---|---|---|---|---|---|---|---|
| | 5° C. | | | 37° C. | | | 5° C. | | | 37° C. | | |
| Sample | % | % | % | % | % | % | % | % | % | % | % | % |
| # | HMW | Main | LMW | HMW | Main | LMW | HMW | Main | LMW | HMW | Main | LMW |
| 1 | 1.9 | 97.2 | 0.9 | 4.1 | 94.9 | 0.9 | 2.7 | 96.3 | 1.0 | 6.4 | 92.4 | 1.2 |
| 2 | 1.1 | 97.9 | 1.0 | 2.4 | 96.4 | 1.2 | 1.4 | 97.5 | 1.1 | 3.6 | 94.9 | 1.5 |
| 3 | 1.2 | 97.6 | 1.2 | 2.2 | 96.3 | 1.4 | 1.5 | 97.4 | 1.1 | 4.0 | 94.3 | 1.8 |
| 4 | 1.0 | 97.9 | 1.1 | 6.2 | 91.6 | 2.3 | 1.3 | 97.6 | 1.1 | 30.8 | 64.9 | 4.3 |
| 5 | 0.9 | 97.9 | 1.1 | 2.1 | 96.4 | 1.5 | 1.1 | 97.9 | 1.0 | 8.9 | 88.8 | 2.3 |
| 6 | 1.2 | 97.8 | 1.0 | 2.5 | 96.5 | 1.0 | 1.5 | 97.5 | 1.0 | 7.2 | 90.9 | 1.8 |
| 7 | 0.8 | 98.2 | 1.0 | 2.3 | 95.7 | 1.9 | 0.9 | 98.0 | 1.1 | 28.7 | 67.0 | 4.3 |
| 8 | 1.0 | 97.7 | 1.4 | 1.6 | 91.7 | 6.7 | 1.3 | 97.1 | 1.6 | 2.6 | 86.0 | 11.5 |
| 9 | 1.0 | 97.9 | 1.1 | 2.0 | 96.6 | 1.5 | 1.3 | 97.6 | 1.1 | 4.2 | 93.7 | 2.1 |
| 10 | 1.0 | 97.7 | 1.2 | 2.1 | 96.2 | 1.7 | 1.3 | 97.6 | 1.1 | 6.2 | 91.5 | 2.3 |
| 11 | 1.2 | 97.5 | 1.3 | 2.3 | 96.2 | 1.5 | 1.6 | 97.3 | 1.1 | 5.2 | 92.8 | 2.0 |
| 12 | 1.3 | 97.7 | 0.9 | 3.3 | 95.6 | 1.1 | 1.9 | 97.1 | 1.0 | 8.7 | 89.6 | 1.7 |
| 13 | 1.0 | 98.1 | 0.9 | 2.2 | 96.8 | 1.0 | 1.3 | 97.7 | 1.0 | 8.9 | 89.3 | 1.8 |
| 14 | 1.1 | 97.6 | 1.3 | 2.0 | 96.5 | 1.5 | 1.2 | 97.7 | 1.0 | 9.2 | 88.4 | 2.4 |
| 15 | 1.1 | 97.9 | 1.0 | 2.3 | 96.3 | 1.4 | 1.5 | 97.5 | 1.0 | 4.6 | 93.5 | 1.9 |
| 16 | 1.1 | 97.6 | 1.3 | 2.6 | 95.2 | 2.2 | 1.1 | 97.8 | 1.1 | 10.3 | 86.6 | 3.1 |
| 17 | 1.0 | 97.9 | 1.1 | 3.8 | 94.1 | 2.1 | 1.3 | 97.6 | 1.1 | 16.2 | 80.4 | 3.4 |
| 18 | 1.2 | 97.6 | 1.1 | 3.2 | 95.2 | 1.6 | 1.7 | 97.2 | 1.1 | 7.9 | 89.6 | 2.5 |
| 19 | 0.9 | 97.9 | 1.2 | 2.2 | 96.3 | 1.5 | 1.1 | 97.9 | 1.0 | 12.2 | 85.5 | 2.3 |
| 20 | 1.7 | 97.4 | 0.9 | 3.7 | 95.3 | 1.0 | 2.3 | 96.7 | 1.1 | 8.1 | 90.2 | 1.8 |
| 21 | 1.2 | 97.8 | 1.0 | 2.5 | 96.4 | 1.1 | 1.6 | 97.4 | 1.1 | 7.7 | 90.3 | 1.9 |
| 22 | 0.9 | 97.9 | 1.2 | 3.5 | 94.5 | 2.0 | 1.2 | 97.7 | 1.1 | 10.5 | 86.8 | 2.8 |
| 23 | 1.0 | 98.0 | 1.0 | 2.7 | 95.9 | 1.4 | 1.3 | 97.5 | 1.2 | 7.9 | 89.9 | 2.1 |
| 24 | 0.8 | 98.2 | 0.9 | 2.9 | 95.7 | 1.4 | 1.0 | 97.9 | 1.1 | 12.9 | 84.7 | 2.5 |
| 25 | 1.2 | 97.8 | 0.9 | 2.9 | 95.9 | 1.2 | 1.6 | 97.3 | 1.1 | 7.2 | 91.0 | 1.7 |
| 26 | 0.8 | 98.0 | 1.2 | 4.3 | 93.7 | 2.0 | 0.9 | 98.0 | 1.0 | 31.7 | 64.0 | 4.3 |
| 27 | 0.9 | 98.2 | 0.9 | 4.5 | 94.1 | 1.4 | 1.3 | 97.7 | 1.1 | 34.9 | 61.4 | 3.7 |
| 28 | 1.3 | 97.7 | 1.0 | 2.8 | 95.9 | 1.2 | 1.5 | 97.4 | 1.0 | 8.3 | 89.6 | 2.1 |
| 29 | 1.2 | 97.8 | 1.0 | 2.4 | 96.5 | 1.1 | 1.6 | 97.3 | 1.1 | 3.4 | 95.2 | 1.4 |
| 30 | 1.0 | 98.0 | 0.9 | 2.6 | 95.9 | 1.4 | 1.3 | 97.6 | 1.1 | 8.1 | 89.7 | 2.1 |
| 31 | 1.2 | 97.8 | 1.0 | 2.3 | 96.4 | 1.2 | 1.4 | 97.6 | 1.1 | 4.3 | 94.0 | 1.7 |
| 32 | 1.2 | 97.9 | 0.9 | 2.6 | 96.4 | 1.0 | 1.7 | 97.3 | 1.0 | 6.4 | 92.0 | 1.6 |
| 33 | 1.0 | 98.0 | 1.0 | 3.6 | 94.9 | 1.5 | 1.3 | 97.5 | 1.2 | 30.4 | 66.1 | 3.5 |
| 34 | 1.0 | 98.0 | 1.1 | 2.1 | 96.4 | 1.5 | 1.3 | 97.6 | 1.1 | 20.0 | 77.4 | 2.6 |
| 35 | 1.1 | 98.0 | 0.9 | 2.3 | 96.6 | 1.1 | 1.3 | 97.7 | 1.0 | 10.4 | 87.3 | 2.3 |
| 36 | 1.1 | 98.0 | 1.0 | 2.7 | 95.9 | 1.3 | 1.5 | 97.4 | 1.1 | 6.8 | 91.2 | 2.1 |
| 37 | 0.8 | 98.0 | 1.2 | 1.5 | 96.9 | 1.6 | 1.0 | 98.0 | 1.0 | 3.4 | 94.5 | 2.0 |

# = samples #

cIEF: The cIEF (capillary Isoelectric Focusing) results at weeks 4 and 7 (5° C., 37° C.) time points are shown in Table 9.

TABLE 9
charge variants

| | 4 weeks | | | | | | 8 weeks | | | | | |
|---|---|---|---|---|---|---|---|---|---|---|---|---|
| | 5° C. | | | 37° C. | | | 5° C. | | | 37° C. | | |
| | % | % | % | % | % | % | % | % | % | % | % | % |
| # | Acidic | Main | Basic | Acidic | Main | Basic | Acidic | Main | Basic | Acidic | Main | Basic |
| 1 | 35.9 | 51.2 | 12.9 | 49.3 | 38.7 | 12.0 | 37.1 | 50.3 | 12.5 | 60.8 | 28.2 | 11.0 |
| 2 | 37.2 | 48.9 | 13.9 | 46.3 | 39.4 | 14.2 | 36.0 | 50.8 | 13.2 | 56.6 | 30.5 | 12.9 |
| 3 | 36.7 | 50.4 | 13.0 | 51.8 | 37.1 | 11.1 | 37.2 | 50.4 | 12.4 | 68.0 | 23.4 | 8.6 |
| 4 | 34.8 | 51.9 | 13.3 | 35.6 | 35.6 | 28.8 | 37.3 | 49.3 | 13.4 | 36.6 | 16.8 | 46.6 |
| 5 | 36.8 | 50.1 | 13.1 | 47.2 | 38.3 | 14.5 | 36.5 | 49.9 | 13.6 | 50.8 | 19.3 | 29.9 |
| 6 | 36.9 | 50.4 | 12.8 | 54.2 | 34.3 | 11.5 | 37.9 | 49.9 | 12.2 | 75.0 | 13.6 | 11.3 |
| 7 | 36.5 | 50.9 | 12.6 | 42.1 | 38.8 | 19.1 | 36.8 | 50.2 | 13.0 | 31.2 | 14.2 | 54.6 |
| 8 | 35.9 | 50.8 | 13.3 | 43.1 | 42.7 | 14.2 | 37.9 | 49.4 | 12.7 | 54.3 | 31.7 | 14.0 |
| 9 | 35.9 | 51.9 | 12.2 | 44.0 | 41.7 | 14.3 | 37.3 | 50.4 | 12.3 | 55.7 | 30.6 | 13.6 |
| 10 | 36.8 | 50.0 | 13.2 | 43.9 | 40.8 | 15.3 | 37.1 | 50.6 | 12.3 | 55.2 | 28.7 | 16.1 |
| 11 | 35.9 | 51.3 | 12.9 | 51.4 | 36.5 | 12.2 | 36.5 | 51.0 | 12.5 | 69.7 | 21.6 | 8.7 |
| 12 | 36.3 | 50.4 | 13.3 | 46.4 | 39.2 | 14.4 | 36.2 | 50.5 | 13.3 | 61.2 | 25.5 | 13.3 |
| 13 | 34.4 | 52.1 | 13.4 | 45.8 | 40.2 | 14.1 | 37.6 | 49.8 | 12.5 | 63.8 | 24.1 | 12.1 |
| 14 | 36.7 | 49.7 | 13.6 | 53.9 | 35.3 | 10.8 | 38.1 | 49.0 | 13.0 | 82.6 | 11.1 | 6.4 |
| 15 | 34.9 | 52.0 | 13.0 | 41.9 | 42.6 | 15.5 | 36.7 | 50.0 | 13.3 | 50.7 | 34.0 | 15.3 |
| 16 | 36.1 | 50.2 | 13.6 | 40.0 | 39.4 | 20.6 | 37.4 | 49.3 | 13.3 | 41.3 | 26.2 | 32.4 |
| 17 | 35.7 | 51.1 | 13.2 | 38.4 | 39.4 | 22.2 | 36.8 | 49.6 | 13.5 | 37.3 | 24.2 | 38.5 |
| 18 | 37.9 | 49.6 | 12.5 | 56.8 | 32.3 | 10.9 | 38.9 | 48.8 | 12.4 | 74.7 | 18.1 | 7.2 |
| 19 | 35.4 | 51.6 | 13.0 | 46.6 | 40.9 | 12.5 | 37.9 | 48.7 | 13.4 | 52.5 | 22.5 | 25.0 |
| 20 | 36.5 | 50.6 | 12.9 | 51.6 | 37.3 | 11.1 | 37.9 | 49.2 | 12.8 | 75.8 | 16.7 | 7.5 |
| 21 | 36.3 | 50.7 | 13.1 | 49.5 | 38.3 | 12.2 | 37.6 | 49.2 | 13.2 | 75.2 | 17.6 | 7.1 |
| 22 | 35.6 | 51.3 | 13.1 | 36.7 | 42.4 | 20.9 | 37.5 | 49.9 | 12.7 | 37.3 | 29.9 | 32.8 |
| 23 | 35.8 | 50.8 | 13.4 | 41.4 | 39.7 | 18.9 | 37.6 | 49.8 | 12.7 | 45.5 | 27.3 | 27.1 |
| 24 | 35.6 | 50.6 | 13.8 | 43.4 | 35.4 | 21.2 | 37.5 | 49.0 | 13.5 | 42.0 | 19.2 | 38.8 |
| 25 | 37.5 | 49.5 | 13.0 | 47.7 | 39.0 | 13.3 | 39.5 | 48.0 | 12.5 | 66.8 | 25.1 | 8.1 |
| 26 | 36.3 | 50.6 | 13.1 | 38.7 | 40.9 | 20.4 | 36.2 | 50.8 | 13.1 | 30.6 | 15.4 | 54.0 |
| 27 | 35.0 | 51.4 | 13.7 | 42.5 | 38.1 | 19.4 | 38.6 | 48.0 | 13.5 | 35.7 | 13.2 | 51.1 |
| 28 | 35.4 | 52.1 | 12.5 | 50.8 | 37.2 | 12.0 | 37.4 | 49.5 | 13.1 | 68.3 | 11.3 | 20.5 |
| 29 | 35.9 | 51.7 | 12.4 | 49.4 | 38.9 | 11.7 | 39.3 | 48.3 | 12.4 | 64.9 | 24.9 | 10.2 |
| 30 | 35.3 | 51.0 | 13.7 | 40.5 | 40.6 | 18.9 | 36.4 | 49.7 | 13.9 | 46.9 | 27.6 | 25.4 |
| 31 | 37.0 | 50.2 | 12.9 | 49.8 | 38.1 | 12.1 | 38.1 | 49.4 | 12.5 | 65.7 | 24.6 | 9.7 |
| 32 | 35.9 | 51.1 | 12.9 | 45.0 | 41.5 | 13.5 | 37.9 | 49.5 | 12.6 | 63.2 | 26.0 | 10.7 |
| 33 | 35.8 | 50.3 | 13.9 | 43.0 | 37.0 | 20.0 | 38.1 | 48.6 | 13.3 | 63.9 | 24.7 | 11.4 |
| 34 | 37.0 | 49.5 | 13.4 | 44.4 | 40.3 | 15.3 | 37.9 | 49.1 | 13.0 | 51.3 | 18.2 | 30.5 |
| 35 | 36.8 | 50.6 | 12.6 | 54.4 | 33.9 | 11.7 | 38.3 | 48.9 | 12.8 | 78.3 | 11.9 | 9.9 |
| 36 | 35.2 | 51.8 | 13.0 | 42.4 | 38.6 | 19.0 | 37.9 | 48.6 | 13.5 | 47.3 | 27.5 | 25.2 |
| 37 | 36.3 | 50.9 | 12.8 | 43.2 | 40.4 | 16.5 | 39.1 | 47.8 | 13.1 | 50.9 | 33.0 | 16.1 |

The primary aim of the pre-formulation development studies presented in examples 1 and 2 was to identify formulation components that provide optimal chemical and physical stability of an anti-TG2 antibody. Altogether, the available data indicated that the optimal formulations would include citrate or histidine buffers with pH ranges of 5.5 to 6.5 in combination with either 250 mM glycine or 150 mM NaCl.

The SEC and iCE results obtained during the DoE study were reanalysed using the JMP software (data not shown). A comparison of the JMP suggested preferred formulations from each data set was performed (SEC data set at both 5° C. and 37° C. for % of monomer, % of HMWS and % of LMWS and iCE data set at both 5° C. and 37° C. for % of Main peak, % of acidic species and % of basic species). All in all, from this statistical analysis it appeared that:
both citrate and histidine had very good buffering properties, with a slight preference towards histidine,
a pH of 6.5 was preferred for citrate as the buffering agent and a pH of 5.0-5.5 for histidine,
a buffer strength of 50 mM was most desirable,
both NaCl and glycine provided good results,
the advantage of adding a surfactant was not clear.

### Example 3—Long Term (12 Months) Stability Study on Selected Formulations

Based on the above examples, four formulations were chosen for long term studies (all containing 100 mg/ml of anti-TG2 antibody mAb1):
F1: 50 mM histidine, 250 mM glycine, pH5.5;
F2: 50 mM citrate, 250 mM glycine, 0.06% w/v PS80, pH 5.6;
F3: 50 mM histidine, 150 mM NaCl, pH5.0;
F4: 50 mM histidine, 250 mM glycine, 0.05% w/v PS80, pH5.5.

Four storage conditions were tested: −60° C. (data not shown), 2-8° C., 25° C. and 40° C.

Appearance: During the study no significant aspect change (particles, precipitate, colour, . . . ) had been observed with time. The only minor observation made, was a discolouring, of liquid formulation slowly turning slight yellow along the stability at 40° C. and 25° C. Sample thawed from the ≤−60° C. storage condition could appear very slightly cloudy due to suspended micro-bubbles.

Protein Concentration and pH (data not shown): No significant variation of concentration was observed along the study. Similarly, stable pH was observed across formulations during the study.

Viscosity and Osmolality: Both viscosity and osmolality were in acceptable ranges, respectively between 2.3 and 3.5 cP and between 335 and 385 mOsm/kg water (data not shown).

SEC results: No significant change could be observed in the HMWS % for sample stored at ≤−60° C. As shown in Tables 10 to 15, the increase of the HMWS % over time appeared linear up to 3 months when samples were stored at 2-8° C., 25° C./60% RH and 40° C./75% RH. After those 3 months the HMWS % tend to be stable. The rate of HMWS % per quarter (slope×3) was calculated based on the slope (rate/month) of the results measured on the first three months for samples stored at 2-8° C., 25° C./60% RH, 40° C./75% RH (data not shown). As a summary, at 2-8° C. and 25° C./60% RH, the histidine buffer formulations were the most stable over time. No significant difference between the formulation histidine/glycine or histidine/NaCl could be observed. The addition of the PS80 in the formulation histidine/glycine did not show an evident added value in the stability of the molecule. At 40° C./75% RH, the citrate buffer formulation was the most stable.

TABLE 10
Size Exclusion Chromatography-Monomer %

| | | 2-8° C. | | | | | 25° C./60% RH | | | | | 40° C./75% RH | | |
|---|---|---|---|---|---|---|---|---|---|---|---|---|---|---|
| Id | 0w | 1 m | 2 m | 3 m | 6 m | 12 m | 1 m | 2 m | 3 m | 6 m | 12 m | 1 m | 2 m | 3 m |
| F1 | 96.2 | 95.1 | 96.5 | 96.9 | 95.9 | 95.8 | 94.5 | 96.2 | 96.3 | 94.1 | 94.0 | 92.7 | 92.1 | 89.3 |
| F2 | 95.7 | 94.2 | 95.9 | 96.1 | 95.3 | 95.3 | 93.9 | 95.5 | 95.7 | 93.8 | 94.1 | 92.7 | 93.4 | 92.4 |
| F3 | 96.3 | 94.7 | 96.6 | 97.0 | 96.0 | 96.0 | 94.8 | 96.3 | 96.3 | 93.9 | 93.3 | 88.3 | 86.0 | 80.6 |
| F4 | 96.0 | 94.9 | 96.4 | 96.8 | 95.8 | 95.8 | 94.3 | 96.0 | 96.1 | 94.5 | 93.7 | 91.9 | 91.2 | 87.8 |

TABLE 11
Size exclusion Chromatography-HMWS %

| | | 2-8° C. | | | | | 25° C./60% RH | | | | | 40° C./75% RH | | |
|---|---|---|---|---|---|---|---|---|---|---|---|---|---|---|
| Id | 0w | 1 m | 2 m | 3 m | 6 m | 12 m | 1 m | 2 m | 3 m | 6 m | 12 m | 1 m | 2 m | 3 m |
| F1 | 2.0 | 2.1 | 2.2 | 2.3 | 2.4 | 2.3 | 2.3 | 2.6 | 2.8 | 3.1 | 3.7 | 5.9 | 8.5 | 3.7 |
| F2 | 2.4 | 2.6 | 2.7 | 3.0 | 3.0 | 2.8 | 2.8 | 3.2 | 3.4 | 3.5 | 3.7 | 4.9 | 5.9 | 3.7 |
| F3 | 1.9 | 2.0 | 2.1 | 2.1 | 2.3 | 2.2 | 2.2 | 2.5 | 2.8 | 3.2 | 7.8 | 11.6 | 16.4 | 7.8 |
| F4 | 2.1 | 2.2 | 2.3 | 2.4 | 2.5 | 2.4 | 2.4 | 2.6 | 2.9 | 3.2 | 4.3 | 6.6 | 9.8 | 4.3 |

TABLE 12
Size Exclusion Chromatography-LMWS %

| | | 2-8° C. | | | | | 25° C./60% RH | | | | | 40° C./75% RH | | |
|---|---|---|---|---|---|---|---|---|---|---|---|---|---|---|
| Id | 0w | 1 m | 2 m | 3 m | 6 m | 12 m | 1 m | 2 m | 3 m | 6 m | 12 m | 1 m | 2 m | 3 m |
| F1 | 1.8 | 2.8 | 1.3 | 0.8 | 1.7 | 1.9 | 3.2 | 1.3 | 1.0 | 2.7 | 2.4 | 3.7 | 2.1 | 2.2 |
| F2 | 1.9 | 3.2 | 1.4 | 0.9 | 1.7 | 1.9 | 3.3 | 1.3 | 0.9 | 2.7 | 2.3 | 3.7 | 1.7 | 1.7 |
| F3 | 1.8 | 3.4 | 1.3 | 0.8 | 1.7 | 1.8 | 3.0 | 1.3 | 0.9 | 2.9 | 3.1 | 3.9 | 2.5 | 3.0 |
| F4 | 1.9 | 3.0 | 1.3 | 0.8 | 1.8 | 1.9 | 3.4 | 1.4 | 1.1 | 2.3 | 2.7 | 3.8 | 2.2 | 2.4 |

iCE results: No significant change of main species could be observed when samples were stored at ≤−60° C. and 2-8° C. At 25° C./60% RH and 40° C./75° RH, histidine buffer formulations appeared as the most stable and the addition of PS80 did not show any added value to the stability of the formulation.

TABLE 13
ICE-Main Species

| | | 2-8° C. | | | | | 25° C./60% RH | | | | | 40° C./75% RH | | |
|---|---|---|---|---|---|---|---|---|---|---|---|---|---|---|
| Id | 0w | 1 m | 2 m | 3 m | 6 m | 12 m | 1 m | 2 m | 3 m | 6 m | 12 m | 1 m | 2 m | 3 m |
| F1 | 52.3 | 53.0 | 51.5 | 52.1 | 53.4 | 50.2 | 52.5 | 48.8 | 47.1 | 52.4 | ND | 41.3 | 31.7 | 19.3 |
| F2 | 51.8 | 51.3 | 50.0 | 51.0 | 52.4 | 49.8 | 50.2 | 46.6 | 39.7 | 52.5 | ND | 28.5 | 14.3 | 6.3 |
| F3 | 52.6 | 52.7 | 52.0 | 51.8 | 53.6 | 51.8 | 51.4 | 42.0 | 47.0 | 53.5 | ND | 35.0 | 27.4 | 12.1 |
| F4 | 53.1 | 52.4 | 50.6 | 50.6 | 52.6 | 50.4 | 51.0 | 46.4 | 46.7 | 53.6 | ND | 40.8 | 30.7 | 12.7 |

TABLE 14
ICE-Acidic species %

| | | 2-8° C. | | | | | 25° C./60% RH | | | | | 40° C./75% RH | | |
|---|---|---|---|---|---|---|---|---|---|---|---|---|---|---|
| Id | 0w | 1 m | 2 m | 3 m | 6 m | 12 m | 1 m | 2 m | 3 m | 6 m | 12 m | 1 m | 2 m | 3 m |
| F1 | 37.1 | 36.8 | 36.8 | 37.5 | 36.6 | 36.2 | 36.6 | 39.2 | 41.0 | 37.3 | ND | 45.9 | 53.7 | 64.1 |
| F2 | 37.9 | 38.5 | 38.3 | 38.3 | 37.5 | 37.0 | 39.7 | 42.3 | 50.2 | 38.2 | ND | 62.7 | 79.6 | 87.6 |
| F3 | 36.8 | 36.3 | 37.1 | 37.2 | 36.3 | 34.5 | 36.7 | 46.2 | 39.3 | 35.5 | ND | 36.8 | 41.0 | 46.5 |
| F4 | 36.6 | 38.0 | 37.6 | 38.5 | 37.1 | 35.6 | 37.9 | 39.9 | 41.3 | 36.0 | ND | 46.4 | 54.2 | 57.8 |

TABLE 15
ICE-Basic species %

| | | 2-8° C. | | | | | 25° C./60% RH | | | | | 40° C./75% RH | | |
|---|---|---|---|---|---|---|---|---|---|---|---|---|---|---|
| Id | 0w | 1 m | 2 m | 3 m | 6 m | 12 m | 1 m | 2 m | 3 m | 6 m | 12 m | 1 m | 2 m | 3 m |
| F1 | 10.5 | 10.2 | 11.7 | 10.4 | 10.0 | 13.6 | 10.9 | 12.0 | 11.9 | 10.4 | ND | 12.8 | 14.6 | 16.6 |
| F2 | 10.3 | 10.3 | 11.7 | 10.7 | 10.1 | 13.2 | 10.1 | 11.1 | 10.1 | 9.3 | ND | 8.8 | 6.1 | 6.2 |
| F3 | 10.6 | 11.0 | 11.0 | 11.1 | 10.1 | 13.7 | 11.9 | 11.8 | 13.7 | 11.0 | ND | 28.3 | 31.6 | 41.4 |
| F4 | 10.3 | 9.6 | 11.8 | 11.0 | 10.2 | 14.0 | 11.1 | 13.7 | 12.0 | 10.4 | ND | 12.9 | 15.1 | 29.5 |

### Example 4—Additional Long Term 12 to 48 Months) Stability Study

Based on the results of example 3, although the four tested formulations were very stable, storage of F1 has been tested at long term (up to 4 years) at 2-8° C. Stability has been further assessed based on osmolality, charge variants, aggregates and fragmentation.

TABLE 16
long term stability at 2-8° C.

| | Acceptance | | | | | | | | | | |
|---|---|---|---|---|---|---|---|---|---|---|---|
| Test | criteria | 0M | 1M | 2M | 3M | 6M | 12M | 18M | 24M | 36M | 48M |
| Osmolality | ≥320 | 345 | 343 | 342 | 342 | 339 | 343 | 345 | 344 | 348 | 344 |
| (mOsm/Kg) | | | | | | | | | | | |
| pH | 5.5 ± 0.2 | 5.5 | 5.4 | 5.5 | 5.6 | 5.5 | 5.5 | 5.5 | 5.5 | 5.4 | 5.5 |
| % Monomer | ≥90.0 | 97.2 | 97.4 | 97.3 | 97.2 | 97.9 | 97.8 | 97.3 | 97.2 | 97.0 | 97.0 |
| (SE-HPLC) | | | | | | | | | | | |
| % HMWS | ≤5.0 | 1.3 | 1.5 | 1.6 | 1.5 | 1.6 | 1.8 | 1.8 | 2.0 | 2.4 | 2.2 |
| % acidic | ≤40.0 | 34.1 | 32.9 | 33.2 | 33.6 | 32.5 | 34.8 | 36.9 | 35.0 | 35.3 | 38.4 |
| species | | | | | | | | | | | |
| % main | ≥50.0 | 57.2 | 58.0 | 57.2 | 57.0 | 57.7 | 56.0 | 53.7 | 54.5 | 54.4 | 52.2 |
| peak | | | | | | | | | | | |
| % basic | ≤20.0 | 8.7 | 9.1 | 9.5 | 9.4 | 9.8 | 9.2 | 9.4 | 10.5 | 10.3 | 9.5 |
| species | | | | | | | | | | | |
| Protein cc | 90-110% | 98.5 | 98.4 | 97.3 | 97.3 | 98.6 | 97.9 | 98.6 | 98.6 | 100.3 | 99.1 |
| (mg/mL) | | | | | | | | | | | |
| Potency by | 80-120% | 102 | 98 | 100 | 102 | 102 | 86 | 101 | 107 | 86 | 98 |
| Elisa | | | | | | | | | | | |

The data show that the selected formulation (F1) was stable over time for more than 36 months (and up to 48 months), at temperatures ranged between 2 and 8° C.

### Overall Conclusions

It has surprisingly been shown that anti-TG2 antibodies can be stabilised in presence of either glycine or NaCl. The most stable formulations comprising 10% of anti-TG2 antibody were 1) F1: 50 mM histidine, 250 mM glycine, pH5.5 and 2) F2: 50 mM citrate, 250 mM glycine, 0.06% w/v PS80, pH6.5 or 5.6. Formulations comprising 150 mM NaCl instead of 250 mM glycine were also very promising. The formulation F1 was studied at long term. In particular it was shown that it was very stable over time for up to 36-48 months, at temperatures ranged between 2 and 8° C. In view of the promising results over 12 months at 25° C. for this formulation, it is anticipated that this stability extends at longer term. F2 to F4 could also be good alternatives.

### REFERENCES

1) WO2006100679
2) WO2012146901
3) WO2013175229

1. A stable liquid formulation comprising an anti-TG2 antibody, a buffer keeping the pH between about 5.0 and 7.0, and a stabilizer selected from the group consisting of glycine or NaCl.

2. The stable liquid formulation according to claim 1, wherein the buffer is a histidine or a citrate buffer.

3. The stable liquid formulation according to claim 2, wherein the histidine buffer keeps the pH at or about 5.5±0.2 or wherein the citrate buffer keeps the pH at or about 6.5±0.2.

4. The stable liquid formulation according to claim 1, wherein the concentration of the buffer is at or about 10 to 100 mM, preferably 20 to 80 or even preferably 40 to 60 mM.

5. The stable liquid formulation according to claim 1, wherein the stabilizer is glycine and is in an amount from about 150 mM to 350 mM, preferably 200 to 300 mM or even preferably 220 to 280 mM.

6. The stable liquid formulation according to claim 1, wherein the stabilizer is NaCl and is in an amount from about 100 mM to 200 mM or preferably 125 to 175 mM.

7. The stable liquid formulation according to claim 1, further optionally comprising a polysorbate surfactant.

8. The stable liquid formulation according to claim 7, wherein the concentration of the polysorbate surfactant is at or about 0.01 to 0.5 mg/mL.

9. The stable liquid formulation according to claim 1, wherein the concentration of the anti-TG2 antibody is from about 10 mg/mL to about 200 mg/mL, preferably from about 30 mg/mL to about 180 mg/mL or preferably from about 50 mg/mL to about 150 mg/mL.

10. The stable liquid formulation according to claim 1, wherein the formulation comprises about 100 mg/mL of the anti-TG2 antibody, about 50 mM histidine buffer which keep the pH at or about 5.5, about 250 mM of glycine or about 150 mM NaCl, and optionally about 0.02-0.06 mg/mL of polysorbate.

11. The stable liquid formulation according to claim 1, wherein the formulation comprises about 100 mg/mL of the anti-TG2 antibody, about 50 mM citrate buffer which keep the pH at or about 6.5, about 250 mM of glycine or about 150 mM NaCl, and optionally about 0.02-0.06 mg/mL of polysorbate.

12. The stable liquid formulation according to claim 1, wherein the anti-TG2 antibody comprises:
1) a light chain variable domain having the sequence as defined in SEQ ID NO: 1 and a heavy chain variable domain having the sequence as defined in SEQ ID NO:2
2) a light chain variable domain having at least 80% identity or similarity, preferably 90% identity or similarity to the sequence as defined in SEQ ID NO: 1 and a heavy chain variable domain having at least 80% identity or similarity, preferably 90% identity or similarity to the sequence as defined in SEQ ID NO: 2.
3) a light chain having the sequence as defined in SEQ ID NO: 3 and a heavy chain having the sequence as defined in SEQ ID NO: 4; or
4) a light chain having at least 80% identity or similarity, preferably 90% identity or similarity to the sequence as defined in SEQ ID NO: 3 and a heavy chain having at least 80% identity or similarity, preferably 90% identity or similarity to the sequence as defined in SEQ ID NO: 4.

13. A method for manufacturing the stable liquid formulation according to claim 1, comprising the steps of forming a mixture of the anti TG2 antibody together with 1) the histidine or citrate buffer, 2) glycine or NaCl and optionally 3) the optional polysorbate surfactant.

14. An article of manufacture comprising a container comprising the stable liquid formulation according to claim 1.

15. The stable liquid formulation according to claim 1, for use in therapy.

16. A method for treating a disease or disorder by administering the stable liquid formulation according to claim 1.


SEQUENCE LISTING (4 sequences)

SEQ ID NO: 1 - protein, 107 aa, synthetic construct
  REGION 1..107: note = Artificial sequence
DITMTQSPSSLSASVGDRVTITCKASQDINSYLTWFQQKPGKAPKILIYLVNRLVDGVPSRFSGSGSGQDYALTISSLQPEDFATYYCLQYDDFPYTFGQGTKVEIK

SEQ ID NO: 2 - protein, 114 aa, synthetic construct
  REGION 1..114: note = Artificial sequence
EVQLLESGGGLVQPGGSLRLSCAASGFTLSTHAMSWVRQAPGKGLEWVATISSGGRSTYYPDSVKGRFTISRDNSKNTLYLQMNSLRAEDTAVYFCARLISTYWGQGTLVTVSS

SEQ ID NO: 3 - protein, 214 aa, synthetic construct
  REGION 1..214: note = Artificial sequence
DITMTQSPSSLSASVGDRVTITCKASQDINSYLTWFQQKPGKAPKILIYLVNRLVDGVPSRFSGSGSGQDYALTISSLQPEDFATYYCLQYDDFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 441 aa, synthetic construct
  REGION 1..441: note = Artificial sequence
EVQLLESGGGLVQPGGSLRLSCAASGFTLSTHAMSWVRQAPGKGLEWVATISSGGRSTYYPDSVKGRFTISRDNSKNTLYLQMNSLRAEDTAVYFCARLISTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK